(19) United States
(12) Patent Application Publication
(10) Pub. No.: US 2007/0141722 A1
(43) Pub. Date: Jun. 21, 2007

(54) Device, system and method of detecting targets in a fluid sample

(76) Inventors: Stephane Andre Follonier, Pleasant Hill, CA (US); Pierre Francois Indermuhle, Berkeley, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/572,931

(22) PCT Filed: Sep. 22, 2004
(86) PCT No.: PCT/US04/31259

§ 371 Date: Feb. 5, 2007

(30) Foreign Application Priority Data
Sep. 24, 2003 (US) 10670912

(51) Int. Cl. G01N 33/543

(52) U.S. Cl. 436/518

(57) ABSTRACT

The present invention provides a biochemical detection system that comprises an exchangeable cartridge unit with light guiding tubes pre-coated with capture agent(s) and an optical detection unit Upon flowing the liquid or gaseous sample containing the target(s) through the cartridge until the target(s) bind(s) to the capture agent(s) and is (are) detected by the amount of light or the variation of its properties while guided through the tubes. The optical detection unit is comprised of a light emitting element(s), a light connecting element(s) and a light detecting element(s) that delivers the amount of target(s) in the sample under investigation.

### TECHNICAL FIELD

The present invention is directed to devices that can detect targets in fluid samples.

### BACKGROUND

Over the past decade, miniaturization and integration have revolutionized the world of biotechnology, allowing the realization of small sample volume, high throughput and multiplexed assays. From DNA micro-arrays to Elisa 1536-well plates, multiplexed systems appear to be the promising approach for biotechnology. These systems are directed towards highly multiplexed assays with multiple capture agents and targets. However, for clinical diagnostic tests, higher specificity (lower false positive data points), lower multiplexing (<10 capture agents), shorter assay time (30 minutes or less) as well as easier handling are required.

The diagnostic tests available on the market today suffer from one or more of the following disadvantages: long assay time (more than 30 minutes), large sample volume, low throughput, high complexity and especially a lack of modularity. Therefore, there is a strong need for an easy to handle, high sensitivity/selectivity/specificity, lower multiplexing, low cost, low sample volume and high throughput device, which can perform quantitative measurements of target(s) concentration in samples.

### SUMMARY OF INVENTION

Our approach is based on building a device that comprises an exchangeable cartridge unit coupled with a detection system. The cartridge unit is seen as a bundle of short light guiding tubes with pre-coated highly specific capture agents on their inner walls that can be very easily tailored to the customer request. By inserting the desired cartridge unit as well as the sample in the instrument, the user can determine the concentration of a panel of targets of his choice in this sample. Depending on the application, there are a variety of techniques to make the tubes that compose the cartridge light guiding.

Thanks to the flexibility of our approach, the use of the cartridge unit can be expanded from pure biotechnological applications (DNA, proteins) to different functionalities such as chemicals, toxins, viruses and/or bacteria or any other targets in liquid samples for which a capture agent can be engineered. Applications such as water quality monitoring, environmental safety monitoring, rapid diagnostic kits, portable field sensors, integrated point of care sensors are among the many possible applications. Potential users include research institutes, pharmaceutical companies, analysis laboratories as well as point of care customers both in military and in civil applications.

By expanding the exchangeable cartridge unit from a liquid waveguide to a gaseous waveguide (using a photonic bandgap crystal structure), our approach also covers measurements of air born pathogens such as anthrax, SARS or other viruses/bacteria or any target, for which a capture agent can be produced. Applications such as sensor for explosives, environment sensor, air quality sensor or military portable sensors are among many possible applications.

### DISCLOSURE OF THE INVENTION

In one aspect, this invention is directed to a measuring cell, which comprises at least one tube capable of both guiding light and binding a target(s) from a liquid or gaseous sample facilitated by to at least one capture agent immobilized on its inner surface.

This tube(s), that comprises an input opening, an output opening and an inner surface coated with a binding agent(s), is exposed to a sample by flowing, in a regulated manner, the sample into the input opening, through the tube(s) and out from the output opening. The flow of the sample through the tube can be regulated by pressure, gravity, capillary forces or electrophoresis.

The ability of the tube(s) to guide light is generated either by the properties of its inner surface (which may be made of one or more organic or inorganic layer, e.g. in such a way that this layer or these layers builds an optical coating) or through an inherent property of the material used to construct the tube(s). Alternately, the ability of the tube(s) to guide light is a result of features designed within the material building the tube(s) or is a result of features designed within a material surrounding the tube(s). Examples of such tube(s) are hollow fibers and photonic bandgap crystals. Another alternative to generate the ability of the tube to guide light is a the result of the introduction into the tube of a fluid (e.g. a liquid) with a refractive index high enough to render the tube or the tube with its surrounding material a light guide.

The capture agent(s) may be bound directly to the inner surface of the tube(s) (or to one of the layers building it) or bound to an interstitial layer comprised of one or more layers. This layer(s) may contain an additional agent(s) that prevents or retards non-specific adsorption and/or non-specific binding of the target(s) and/or other components of the sample. In another embodiment, the inner surface of the tube is coated with an additional layer, which interacts with the bound target in a way that changes the properties of the light guided through the tube.

In another aspect, this invention is directed to a system that comprises a light emitting element(s), a primary light connecting element(s), a measuring cell as described in the first aspect, a secondary light connecting element(s), a light detecting element(s) and a fluid dispensing element(s). It may also comprise a sample and a disposal reservoir.

In this system, the fluid dispensing element(s) dispenses in a regulated manner the liquid or gaseous sample from the sample reservoir into the measuring cell and from the measuring cell into the disposal reservoir. The light, emitted by the light emitting element(s), is connected to the measuring cell by the primary light connecting element(s). It is guided through this measuring cell and then connected through the secondary light connecting element(s) to the light detecting element(s). The change in the amount or in the properties of the detected light relates to the amount of the target(s) bound to the capture agent(s) on the inner surface of the tube(s) of the measuring cell, or to a change of at least one of its properties.

Examples of the light emitting element(s) are a laser, a Light Emitting Diode, a white light source, a Vertical cavity light emitting laser and an array of those elements. Examples of the light detecting element(s) are a photomultiplier tube, a camera, a photodiode and an array of those elements. Examples of light connecting element(s) are a Brewster angle window, a lenslet array, a grating index coupler, a partially reflecting mirror, a spectral or an intensity filter and a combination of two or more of the connecting elements described above. The light connecting element(s) that may be the same or not, may also be a liquid dispensing element(s). In another embodiment, the light connecting element(s) is integrated in the tube(s) of the measuring cell.

The ability of the tube(s) to guide light is generated either by the properties of its inner surface (which may be made of one or more organic or inorganic layer, e.g. in such away that this layer or these layers build an optical coating) or through an inherent property of the material used to construct the tube(s). Alternately, the ability of the tube(s) to guide light is a result of features designed within the material building the tube(s) or is a result of features designed within a material surrounding the tube(s). Examples of such tube(s) are hollow fibers and photonic bandgap crystals. Another alternative to generate the ability of the tube to guide light is a the result of the introduction into the tube of a fluid (e.g. a liquid) with a refractive index high enough to render the tube or the tube with its surrounding material a light guide.

The capture agent(s) may be bound directly to the inner surface of the tube(s) (or to one of the layers building it) or bound to an interstitial layer comprised of one or more layers. This layer(s) may contain an additional agent(s) that prevents or retards non-specific adsorption and/or non-specific binding of the target(s) and/or other components of the sample. In another embodiment, the inner surface of the tube is coated with an additional layer, which interacts with the bound target in a way that changes the properties of the light guided through the tube.

In a third aspect, this invention is directed to a method for detecting a target(s) in a liquid or gaseous sample. This method comprises the introduction, using the fluid dispensing element(s), of a sample into the measuring cell(s), which comprises at least one tube capable of both guiding light and binding a target(s) from a sample. This method also comprises the step of connecting the light emitted by the light emitting element(s) into the measuring cell using the primary light connecting element(s), wherein the light is then guided through the measuring cell where it interacts with the bound target(s). In addition, it comprises the step of connecting light, by using the secondary light connecting element(s), from the measuring cell(s) to the light detecting element(s). The detection, with the light detecting element(s), of the amount of light or of the variation of the property(ies) of the light that went through the measuring cell allows the determination or the calculation of the amount of target(s) bound to the capture agent(s) on the inner surface of the measuring cell, or of the properties of this target.

The mentioned tube(s) comprises an input opening, an output opening and an inner surface coated with binding agent(s). In this method, the fluid dispensing element(s) dispenses the liquid or gaseous sample into the measuring cell from the sample reservoir and from the measuring cell into the disposal reservoir in a regulated manner.

In another embodiment, the method comprises the introduction, after the sample is introduced to the measuring cell using a fluid dispensing element(s), of one cleaving and/or digesting agent into the at least one measuring cell, using at least one fluid dispensing element, after the at least one target is immobilized on the inner surface of the at least one measuring cell in a first step, and wherein the at least one cleaving and/or digesting agent modifies the structure of the at least one bound target. In yet another embodiment, the method comprises the introduction, after the sample is introduced to the measuring cell using a fluid dispensing element(s), of a second binding agent(s) into the measuring cell that binds to the target(s), which has been captured by the capture agent(s). The second binding agent(s) emits light or absorbs light or has optical properties that enhance detection. The interaction of the target(s) with any agent and/or any layer bound or immobilized on the inner surface of the tube may change the optical properties of either, the second binding agent(s), the bound target(s) or any agent and/or any layer bound or immobilized on the inner surface of the tube. This interaction or the optical properties of the second binding agent(s) changes the amount of light or the property(ies) of the light that went through the measuring cell allowing the determination or the calculation of the amount of target(s) bound to the capture agent(s) on the inner surface of the measuring cell, or of the properties of this target.

In a further embodiment, the method comprises the introduction of an amplification agent(s) to the measuring cell(s), where the amplification agent(s) binds to the second binding agent(s). The amplification agent(s) emits light or absorbs light or has optical properties that enhance detection. The interaction of the target(s) with any agent and/or any layer bound or immobilized on the inner surface of the tube may change the optical properties of either the bound target(s) or any agent and/or any layer bound or immobilized on the inner surface of the tube. This interaction or the optical properties of the amplification agent(s) changes the amount of light or the property(ies) of the light that went through the measuring cell allowing the determination or the calculation of the amount of target(s) bound to the capture agent(s) on the inner surface of the measuring cell, or of the properties of this target.

In another embodiment, the sample undergoes a required number of sample preparation steps before being introduced into the measuring cell.

In yet another embodiment, the method comprises or not a washing step between any immobilization or detection steps.

In another embodiment, an optical fluid (e.g. a liquid) is introduced into the at least one tube of the at least one measuring cell, said optical fluid having a refractive index high enough to render the at least one tube or the at least one tube with its surrounding material a light guide.

In a further embodiment, the optical fluid is introduced at any step of the method or between any step of the method or both.

In yet another embodiment, the optical fluid is kept in the at least one tube of the at least one measuring cell during the time necessary to perform any desired measurement(s).

In a further embodiment, the immobilization times are adequately chosen for each step of each embodiment of the method

### BRIEF DESCRIPTION OF DRAWINGS

FIG. 1: The biochemical detection system comprising an optical detection unit with one light emitting element (101), with one primary light connecting element (102), with one secondary light connecting element (103), and with one light detecting element (104). The system further comprises an exchangeable cartridge unit (105) with light guiding tubes pre-coated with capture agent(s) and one fluid dispensing element (106).

### MODE FOR CARRYING OUT THE INVENTION

The following detailed description illustrates the invention by way of example and not by way of limitation This description will clearly enable one skilled in the art to make and use the invention, and describes several embodiments, adaptations, variations, alternatives and uses of the invention, including what we presently believe is the best mode of carrying out the invention.

The present invention comprises a method to detect targets in fluidic samples and a system enabling the application of this method. The system comprises at least one measuring cell capable of binding targets from a sample; this measuring cell is integrated in an exchangeable cartridge unit, which in turn is coupled to the detection system. These three elements build an extremely sensitive, inexpensive and compact system for quantitative detection of targets in a sample (liquid or gaseous).

The Method

A tube filled with gas or liquid may be turned into an optical waveguide by a specific design of its optical properties or by a specific choice of the optical properties of the fluid. A change in the optical properties of the fluid filling the tube or a change of the properties of the interface between the tube and the fluid may induce a change in the amount or in the characteristics of the guided light. The method described here uses the above principle to detect a target in a fluid sample: the inner surface of the tube is engineered such that the target will be immobilized or bound to this surface when the sample is flown through the tube. The optical characteristics of the target, or of an agent bound to the target (e.g. for specificity or amplification), or the interaction of the target, or of any agent, with the inner surface of the tube, or with any other agent, may generate a variation in the amount or in the properties of the guided light, which can be detected. This variation is proportional to the amount of targets bound to the inner surface of the tube.

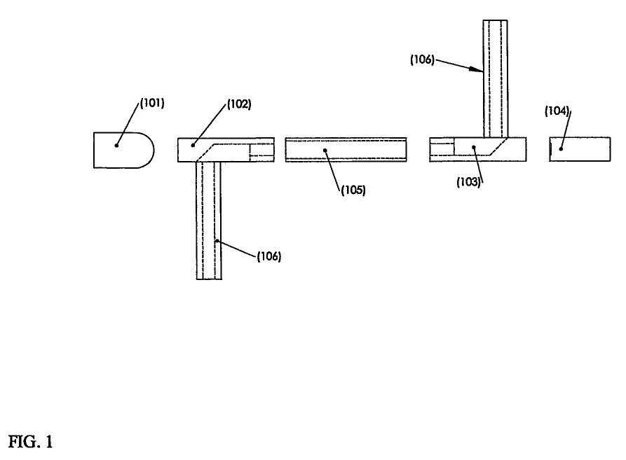

In FIG. 1, a set-up enabling the use of the method described above is schematically represented. The light emitted by a light emitting element(s) (FIG. 1, (101)) is connected to one or more measuring cell (FIG. 1, (105)) through a primary light connecting element(s) (FIG. 1, (102)). The light travels through the measuring cell(s) before being connected out of the measuring cell(s) and into a light detection element(s) FIG. 1, (104)) by a secondary light connecting element(s) (FIG. 1, (103)). The sample of interest is directed to the measuring cell(s) and its flow through the measuring cell(s) is regulated by the fluid dispensing element(s) (FIG. 1, (106)). Upon binding of the target(s) contained in the sample to the capture agent(s) bound to the inner surface of the tube(s) of the measuring cell(s), the amount of light guided through the tube or at least one property of this light is changed proportionally to the amount of target(s) bound to the capture agent(s).

A capture agent is a molecule or a part of a molecule that is capable of binding a target, i.e. capable of immobilizing for a certain period of time another molecule or another part of a molecule contained in a sample. Examples of targets are explosives, pathogens, bacteria, viruses, DNA strands or proteins. Examples of capture agents are molecules/polymers with specific end-groups such as biotin or amine reactive terminals or more complex species such as antibodies, DNA strands. For the method to detect the cleavages of parts of the target(s) (kinase, protease) processes, a cleaving and/or digesting agent can be introduced into the measuring cell after the target is immobilized on the inner surface of the measuring cell in a first step. In order to increase the sensitivity of the detection or to allow for specific types of detections, a second binding agent(s) may interact with the target(s). The second binding agent(s) may also serve as a second filter, by lowering the influence of non-specific binding to the capture agent(s). It may be labeled with a fluorescent dye or with an absorbing molecule such that the interaction of the guided light with this dye or with this molecule results in a change of the properties of the guided light. In order to further increase the sensitivity of the assay, an amplification agent(s) may be bound to the second agent(s) serving a signal amplification purpose and a further filter. Examples of a second binding agent(s) are secondary antibodies conjugated to HRP (horseradish peroxidase) with the corresponding amplification agent being a signal enhancement substrate (e.g. Tetra methyl Benzidine) (Molecular Probes Inc. Eugene, Oreg. 97402). Washing steps may be used to wash off excess of sample, second binding agent(s) or amplification agent(s).

With gaseous samples and with liquid samples, which are partially light transparent, the measurement can occur simultaneously to the sample flow and can run continuously through the measuring cell. Additional automated fluidic devices may allow for additional assay steps for the sample preparation as well as for targets and for agents that require rinsing and/or signal amplification after their immobilization/incubation.

The Measuring Cell

The measuring cell comprises at least one tube, whose walls are coated with at least one specific capture agent to bind at least one specific target from a sample containing known and/or unknown components.

The Tube

The at least one tube has one input and one output openings such that the sample can be introduced into and flown through the tube. The flow through the tube can be regulated through pressure, through capillary forces, through gravity, through electrophoresis, through pumps (Fluidigm Inc. South San Francisco, Calif. 94080), through passive or active valves or through an external flow control device. The sample may be liquid or gaseous.

The at least one tube has also the ability to guide light. In a first example, and thanks to its low refractive index (n<1.33), Teflon AF polymer (Dupont) can be used either as a coating material or as a construction material (Biogeneral, Inc. San Diego, Calif. 92121), to fabricate a tube, which acts like an optical guide. In a second example, a tube fabricated with a photonic bandgap crystal (U.S. Pat. No. 6,571,045 May 27, 2003 Hasegawa et al.), acts like an optical guide when filled with a gas. This is the result of features, designed within the material building the tube or within the material surrounding the tube. In a third example, a tube made out of glass, fused silica or another material becomes light guiding when filled with a fluid having a higher refractive index (Cargille Laboratories, Inc., Cedar Grove, N.Y. 07009) than the effective refractive index of its own material.

The capture agents may be bound directly to the inner surface of the tube through, for example, chemical binding or may be bound indirectly through at least one interstitial layer. Examples of such interstitial layers are polymers (PLL-PEG, silanes, Self-Assembled Monolayers (alcanethiols)). Specifically, for tubes made out of Teflon AF (i.e. for liquid samples), the inner surface of the tubes may be modified by oxygen plasma so that it directly binds the capture agent(s); it may also be coated with an interstitial layer of nitrocellulose or of e.g. Optodex (Arrayon Biotechnology SA, Neuchatel, 2007, Switzerland). For tubes made out of or coated with glass (filled with a fluid with a refractive index high enough to render the tube or the tube with its surrounding material a light guide) or silicon for photonic bandgap crystal, the capture agent may be bound to the tube inner surface by, for example, off-the-shelf silane surface chemistry. For tubes made out of or coated with other materials, e.g. plastics, polymers, any or a combination of the above surface chemistries may be used.

If desired, the inner surface of the at least one tube may also be provided with additional agents that prevents or retards non-specific adsorption and/or non-specific binding of the target and/or of other components of the sample. These additional agents ensure that only the target in the sample binds to the inner surface of the tube and will therefore ensure the specificity of the assay. Examples of those additional agents are PEG chains.

The diameter and the length of the at least one tube depend on the sensitivity and sample volume requested by the application, typically from 5 microns to 1000 microns for the diameter and from 1 mm to 1000 mm for the length For liquid samples, e.g., for strong and specific antibody-antigen interactions, a length of 10 mm for a diameter of 50 microns to 100 microns is a good fit Gaseous samples may require a longer tube (100 nm to 1000 mm) to increase the size of active surface to allow the detection of smaller amounts of targets in the sample. Similarly, the incubation time, i.e. the total time during which the sample is in contact with the capture agent, can be set depending on the type of interaction and the desired sensitivity of the assay, typically between 1 minute and 4 days and preferably between 1 and 30 minutes. Continuous real-time monitoring is also possible.

The Measuring Cell

The measuring cell is an assembly of one or more tubes that are pre-loaded with similar or different capture agents to allow for duplicates or to detect several targets in the same sample or to serve as calibration. Several of such tubes can be held together by integration (Schott Glas, 55122 Mainz, Germany), before or after loading the capture agents. For ease in production, the capture agent loading can be achieved in longer tubes that are cut to size in a second step, ensuring thus the most efficient homogeneity and facilitating the QC/QA process. Finally the measuring cell may be filled with a buffer or a preservation solution and sealed, to prevent any degradation of the active capture agents during the storage and shipping.

The measuring cell may or may not comprise a primary light connecting element(s) and/or a secondary light connecting element(s) and/or a fluid dispensing element(s). These elements may or may not be integrated in the measuring cell. In one example, the at least one measuring cell is provided with one primary light connecting element, one secondary light connecting element and a fluidic element.

The primary light connecting element transmits the light from the at least one light emitting element (belonging to the assay unit (detection system?), see below) into at least one tube of the measuring cell and the secondary light connecting element transmits the light out of the at least one tube of the measuring cell into the at least one light detecting element (of the detection system, see below); also, the fluidic element regulates the flow of the sample through the tube of the measuring cell. More specifically, for liquid samples and tubes made out of Teflon AF, the tube(s), cut to size, is connected to glass Brewster windows that serve the purposes of guiding light into and out of the tube (light connecting elements) as well as guiding the sample into and out of the tube (fluidic dispensing elements).

The primary and secondary light connecting elements may be serving other purposes such as focusing light into the tube (lens, lenslet arrays), such as tailoring the properties of the light (wavelength and intensity filters) or such as reflecting part of the light back into

the tube to allow multiple passes through the tube (partially reflecting mirrors). The fluidic element or part of it may also serve other purposes such as introducing different samples into the tube (second binding agent, amplification agent, buffers), such as regulating the sample flow or such as performing sample preparation, including sample filtering, sample mixing or sample dilution. In one example, the sample flow is controlled via gravity from the input opening of the tube to the output opening of the tube. In addition, the fluidic element may also be used to introduce one or more fluids with a defined refractive index into the tube to control its light guiding properties.

The input and output openings of the tube(s) may be sealed or may be covered with slits to allow an easier handling and protect the content of the measuring cell against any environmental contamination. These seals or slits will break or slide upon insertion of the measuring cell into the detection system unit or at the insertion of the sample into the tube. The measuring cell may also be sealed to preserve the contents integrity until it is used

The measuring cell can be packaged in a user friendly cartridge to be inserted in the detection system

The Detection System

The exchangeable measuring cell (packaged in a cartridge unit) is coupled to a detection system that may be comprised of at least one light emitting element and at least one light detecting element Further light connecting elements may be part of the detection system unit as well as a liquid dispensing unit, a sample reservoir and a waste reservoir.

By inserting the measuring cell, packaged in the exchangeable cartridge unit, into the detection system, the entrance and exit covers slide off the measuring cell(s) and/or the seals of the measuring cell(s) are automatically broken, allowing thus the introduction of the sample into the measuring cell(s). A fluid dispensing element may be used to facilitate the sample flow through the measuring cell(s). The flow of the sample through the measuring cell(s) may be driven by gravity, capillary forces, by electrophoresis or pressure or a combination of these. For gaseous samples, the sample handling system may be comprised of a device that increases the flow through the measuring cells.

The light emitted by the at least one light emitting element is connected to the measuring cell(s) of the exchangeable cartridge unit through the at least one primary light connecting elements. The light travels through the at least one measuring cell before being connected out of the at least one measuring cell and into the at least one light detecting element by the at least one secondary light connecting elements.

When the target contained in the sample binds to the capture agent(s) immobilized on the inner surface of the tube, the change of the amount of light guided through the tube, or the change of at least one property of this light, is measured. For example, the intensity at various wavelengths of the light guided through a measuring cell is changed by the interaction of this light with the target, and/or with the capture agent, and/or with the second binding agent, and/or with the amplification agent bound to the inner surface of the measuring cell(s). Other optical processes such as scattering, or such as the interaction between two of the above species, or between one of the above species and one interstitial layer may also change the amount or at least one property of the transmitted light. The amount of target bound to the capture agents can then be determined or computed by measuring these changes.

The Light Emitting Element

Depending on the application, the at least one light emitting element may be emitting monochromatically or polychromatically in the visible and/or in the infrared and/or in the UV (e.g. Jameco Electronics Belmont, Calif. 94002). It may be a simple light emitting diode or a laser diode or even a white light source (Newport Corporation, Irvine, Calif. 92606) or a Vertical Cavity Surface Emitting Laser. It may be an array of light emitting diodes or lasers or white light sources such that they can be inserted into the tubes. The wavelength(s) of interest may be selected through the at least one primary light connecting element that also serve the purpose of coupling the light into the tube.

The Light Connecting Elements

The primary and secondary light connecting elements serve different purposes such as connecting light from the at least one light emitting element into the at least one measuring cell and out of the at least one measuring cell onto the at least one light detecting element. They can also serve other purposes such as focusing light into separate tubes (lenses, lenslet arrays, Control Optics, Chino, Calif. 91710), such as tailoring the properties of the light (wavelength and intensity filters Newport Corporation, hrvine, Calif. 92606), such as partially reflecting the light back and forth in the tube or such as coupling light into the tube or out of the tube with a grating index coupler. The nature of the primary and the secondary light connecting elements are selected depending on the optical detection process that is used, e.g. fluorescence, absorption, Raman scattering.

The primary and secondary light connecting elements may be a multiplicity of the above described elements for each measuring cell. Besides connecting light into the measuring cell, the purpose of the connecting elements may be to ensure sample handling as well. These light connecting elements may or may not be integrated in the measuring cell.

The Light Detecting Element

The detector, which may be a camera (Jameco Electronics Belmont, Calif. 94002) or a photomultiplier tube or a photodiode or a series of light detecting elements for a multiplicity of measuring cells, monitors the properties and/or the intensity of the light exiting each measuring cell. From the changes at specific wavelengths, of the intensity or of the properties of this light, the processing circuit calculates the concentration in the sample of biologically or chemically relevant targets.

The Fluid Dispensing Element

The fluid dispensing element dispenses the sample to the at least one measuring cell from the sample reservoir and from the at least one measuring cell to the disposal reservoir; the fluid dispensing element may be used to facilitate the sample flow through the measuring cell(s). The flow of the sample through the measuring cell(s) may be driven by gravity, capillary forces, by electrophoresis or pressure or a combination of these. For gaseous samples, the sample handling system may be comprised of a device that increases the flow through the measuring cells. The fluid dispensing element may also be serving other purposes such as introducing different solutions into the tube (secondary binding agent, amplification agent, buffer, optical fluid), such as regulating the sample flow or such as performing sample preparation, including filtering, mixing or sample dilution. In one example, the sample flow is controlled via capillarity from the input opening of the tube to the output opening of the tube. Furthermore, the fluid dispensing element can be used to fill the cell(s) with an index fluid having a refractive index high enough to render the tube or the tube with its surrounding material a light guide.

What is claimed is:

1. A measuring cell comprising at least one tube capable of guiding light, wherein the tube comprises
a) an input opening,
b) an output opening and
c) an inner surface coated with at least one binding agent capable of binding at least one target from a sample,
wherein the inner surface of the at least one tube is exposed to a fluid sample by flowing the sample into the input opening, through the tube and out from the output opening.

2. The measuring cell of claim 1, wherein the sample is liquid or gaseous.

3. The measuring cell of claim 1, wherein the flow of the sample can be regulated.

4. The measuring cell of claim 3, wherein the flow of the sample is regulated by pressure or by gravity or by capillary forces or by electrophoresis.

5. The measuring cell of claim 1, wherein the ability of the tube to guide light is imparted by
a) the structure or the physical properties of the inner surface of the tube,
b) an inherent property of the material used to construct the tube,
c) features designed within the material building the tube,
d) features designed within a material surrounding the tube, or
e) the introduction into the tube of a fluid (e.g. a liquid) with a refractive index high enough to render the tube or the tube with its surrounding material a light guide.

6. The measuring cell of claim 1, wherein the inner surface of the tube may be composed of one or more layers, which layers can be made of an organic or of an inorganic material, or of a combination of both organic and inorganic materials and/or can work as an optical coating.

7. The measuring cell of claim 1, further comprising a material surrounding the tube, which material by its physical properties or by its structure causes the tube to be a light guiding tube.

8. The measuring cell of claim 1, wherein the tube is either a hollow fiber, a photonic bandgap crystal.

9. The measuring cell of claim 1, wherein the tube is filled with an optical fluid (liquid) having a refractive index high enough to render the tube or the tube with its surrounding material a light guide.

10. The measuring cell of claim 1, wherein the at least one capture agent is directly bound to the inner surface of the tube.

11. The measuring cell of claim 1, further comprising an interstitial layer between the at least one capture agent and the inner surface of the tube, wherein the interstitial layer may be a single layer or a multi-layer.

12. The measuring cell of claim 1, wherein the inner surface of the tube is coated with an additional agent that prevents or retards non-specific adsorption and/or non-specific binding of the target and/or other components of the sample.

13. The measuring cell of claim 1, wherein the inner surface of the tube is coated with an additional layer which interacts with the at least one bound target in such a way that it changes the properties of the light guided through the tube.

14. A system comprising:
a) at least one light emitting element;
b) at least one primary light connecting element;
c) at least one measuring cell comprising at least one tube capable of guiding light, wherein the tube comprises
i. an input opening,
ii. an output opening, and
iii. an inner surface coated with at least one binding agent capable of binding at least one target from a sample,
wherein the inner surface of the at least one tube is exposed to a fluid sample by flowing the sample into the input opening, through the tube and out from the output opening;
d) at least one secondary light connecting element;
e) at least one light detecting element and
f) at least one fluid dispensing element;
wherein the at least one fluid dispensing element dispenses the sample to the at least one measuring cell;
further wherein the light emitted by the at least one light emitting element is transmitted to the at least one measuring cell by at least one primary light connecting element;
further wherein the light guided through the at least one measuring cell is transmitted to the at least one light detecting element by the at least one secondary light connecting element;
further wherein the amount of light or the variation of at least one property of the light detected by the at least one light detecting element relates to the amount or to a change of structure and/or properties of the at least one target bound to the at least one capture agent on the inner surface of the at least one tube of the at least one measuring cell.

15. The system of claim 14, where the at least one light emitting element is selected from the group consisting of:
a) a laser;
b) a Light Emitting Diode;
c) a white light source; and
d) a Vertical Cavity Surface Emitting Laser.

16. The system of claim 14, where the at least one light emitting element is a combination or an array of elements selected from the group consisting of:
a) a laser;
b) a Light Emitting Diode;
c) a white light source; and
d) a Vertical Cavity Surface Emitting Laser.

17. The system of claim 14, where the at least one light detecting element is selected from the group consisting of:
a) a Photomultiplier Tube;
b) a camera; and
c) a photodiode.

18. The system of claim 14, where the at least one light detecting element is a combination or an array of elements selected from the group consisting of:
a) a Photomultiplier Tube;
b) a camera; and
c) a photodiode.

19. The system of claim 14, where the at least one primary and the at least one secondary light connecting elements are independently selected from the group consisting of:
a) an optical window;
b) a lenslet array;
c) a spectral filter;
d) a partially reflecting mirror;
e) an intensity filter; and
f) a grating index coupler.

20. The system of claim 14, where one of the at least one primary and the at least one secondary light connecting element or both are also liquid dispensing elements.

21. The system of claim 14, where one of the at least one primary light connecting element and the at least one secondary light connecting element or both are integrated into the measuring cell.

22. The system of claim 14, where the at least one liquid dispensing element is capable of transferring liquid to and from the at least one measuring cell.

23. The system of claim 14, further comprising at least one sample reservoir.

24. The system of claim 14, further comprising at least one disposal reservoir.

25. The system of claim 14, wherein the sample is liquid or gaseous.

26. The system of claim 14, wherein the flow of the sample is regulated.

27. The system of claim 14, wherein the flow of the sample is regulated by pressure or by gravity or by capillary forces or by electrophoresis.

28. The system of claim 14, wherein the ability of the tube to guide light is imparted by
a) the structure or the physical properties of the inner surface of the tube,
b) an inherent property of the material used to construct the tube,
c) features designed within the material building the tube,
d) features designed within a material surrounding the tube, or
e) the introduction into the tube of a fluid (e.g. a liquid) with a refractive index high enough to render the tube or the tube with its surrounding material a light guide.

29. The system of claim 14, wherein the inner surface of the tube may be composed by one or more layer, which can be made of an organic or made of an inorganic material, or of a combination of both organic and inorganic materials and/or can work as an optical coating.

30. The system of claim 14, wherein the tube is either a hollow fiber, a photonic bandgap crystal.

31. The measuring cell of claim 14, wherein the tube is filled with an optical fluid (liquid) having a refractive index high enough to render the tube or the tube with its surrounding material a light guide.

32. The system of claim 14, wherein the at least one capture agent is directly bound to the inner surface of the tube.

33. The system of claim 14, further comprising an interstitial layer between the at least one capture agent and the inner surface of the tube, wherein the interstitial layer may be a single layer or a multi-layer.

34. The system of claim 14, wherein the inner surface of the tube of the measuring cell is coated with an additional layer that prevents or retards non-specific adsorption and/or non-specific binding of the target and/or other components of the sample.

35. The system of claim 14, wherein the inner surface of the tube is coated with an additional layer which interacts with the at least one bound target in such a way that it changes the properties of the light guided through the tube.

36. A method for detecting a target in a sample, which method comprises:
a) introducing a sample to at least one measuring cell using at least one fluid dispensing element, wherein the measuring cell comprises at least one tube capable of guiding light, wherein the tube comprises:
i. an input opening;
ii. an output opening;
iii. an inner surface coated with at least one binding agent capable of binding at least one target of a sample;
wherein the inner surface of the tube is exposed to a fluid sample by flowing the sample into the input opening, through the tube and out from the output opening;
b) connecting light, from at least one light emitting element, into the at least one measuring cell using at least one primary light connecting element, wherein the light is then guided through the at least one measuring cell where it interacts with the at least one bound target;
c) connecting light, using at least one secondary light connecting element, from at least one measuring cell where it interacted with at least one bound target, to at least one light detecting element;
d) detecting, with at least one light detecting element, the amount of light guided through the tube or the variation of at least one property of the light guided through the tube, wherein the amount of light or the variation of at least one of its properties relates to the amount or to a change of structure and/or properties of the at least one target bound to the at least one capture agent on the inner surface of the at least one tube of the at least one measuring cell;
e) determining or calculating the amount of the at least one target bound to the at least one capture agent.

37. The method of claim 36, wherein the flow of the sample is regulated.

38. The method of claim 36, wherein the flow of the sample is regulated by pressure or by gravity or by capillary forces or by electrophoresis.

39. The method of claim 36, wherein the interaction of the at least one target with any agent and/or any layer bound or immobilized on the inner surface of the tube changes the optical properties of either the bound target or of any agent or any layer bound or immobilized on the inner surface of the tube.

40. The method of claim 36, further comprising the step of washing any unbound target and/or component of the sample from the at least one measuring cell before detecting the guided light.

41. The method of claim 36, wherein the sample undergoes the required number of sample preparation steps before being introduced into the measuring cell.

42. The method of claim 36, wherein the immobilization times are adequately chosen for each step of the method.

43. The method of claim 36, wherein an optical fluid (e.g. a liquid) is introduced into the at least one tube of the at least one measuring cell; said optical fluid having a refractive index high enough to render the at least one tube or the at least one tube with its surrounding material a light guide.

44. The method of claim 43, wherein the optical fluid is introduced at any step of the method or between any step of the method or both.

45. The method of claim 43, wherein the optical fluid is kept in the at least one tube of the at least one measuring cell during the time necessary to perform any desired measurement(s).

46. The method of claim 36, wherein at least one cleaving and/or digesting agent is introduced into the at least one measuring cell, using at least one fluid dispensing element, after the at least one target is immobilized on the inner surface of the at least one measuring cell in a first step, and wherein the at least one cleaving and/or digesting agent modifies the structure of the at least one bound target.

47. The method of claim 36, wherein at least one second binding agent is introduced into the at least one measuring cell, using at least one fluid dispensing element, after the at least one target is immobilized on the inner surface of the at least one measuring cell in a first step, and wherein the at least one second binding agent is captured by the at least one bound target.

48. The method of claim 47, wherein the guided light interacts with either the at least one target or with the at least one second binding agent or any agent or any layer bound or immobilized on the inner surface of the tube before it is detected using the at least one detecting element.

49. The method of claim 47, further comprising a washing step between any immobilization or detection step.

50. The method of claim 47, wherein the at least one second binding has optical properties that enhance detection.

51. The method of claim 47, wherein the at least one second binding agent emits light or absorbs light generated by the at least one light emitting element.

52. The method of claim 47, wherein the interaction of the at least one second binding agent with the at least one bound target and/or with any agent and/or any layer bound or immobilized on the inner surface of the tube changes the optical properties of the second binding agent and/or of the bound target and/or of any agent and/or any layer bound or immobilized on the inner surface of the tube.

53. The method of claim 47, wherein the immobilization times are adequately chosen for each step of the method.

54. The method of claim 47, further comprising the step of introducing at least one amplification agent to the at least one measuring cell, where the amplification agent binds to the at least one second binding agent.

55. The method of claim 54, further comprising a washing step between any immobilization or detection step.

56. The method of claim 54, wherein the at least one second binding agent and/or the at least one amplification agent has optical properties that enhance detection.

57. The method of claim 54, wherein the at least one second binding agent and/or the at least one amplification agent emits light or absorbs light generated by the at least one light emitting element.

58. The method of claim 54, wherein the interaction of the at least one amplification agent with the at least one bound target and/or with any agent and/or any layer bound or immobilized on the inner surface of the tube changes the optical properties of the amplification agent and/or of the bound target and/or of any agent and/or any layer bound or immobilized on the inner surface of the tube.

59. The method of claim 58, wherein the immobilization times are adequately chosen for each step of the method.
